(19) United States
(12) Patent Application Publication
(10) Pub. No.: US 2001/0031741 A1
(43) Pub. Date: Oct. 18, 2001

(54) Methods for treatment of lysosomal storage diseases

(76) Inventors: Robin Ziegler, Sterling, MA (US); Seng Cheng, Wellesley, MA (US); John Marshall, Hopedale, MA (US); Mark Goldberg, Needham, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/777,743

(22) Filed: Feb. 6, 2001

Related U.S. Application Data
(60) Provisional application No. 60/183,296, filed on Feb. 17, 2000.
(60) Provisional application No. 60/260,069, filed on Jan. 5, 2001.

(51) Int. Cl. A61K 48/00; A61K 38/47; A61K 31/663

(52) U.S. Cl. 514/44; 424/94.61; 514/102

(57) ABSTRACT

Methods are disclosed for treatment of patients suffering from accumulation of a metabolite within macrophages, such as in lysosomal storage diseases. The methods comprise treating the patient with a macrophage depleting amount of a bisphosphonate compound, such that apoptosis of macrophages is induced and the metabolite is released into circulation so that the metabolites may be eliminated from the patient. The methods may further include administration of a gene therapy vector for the treatment of lysosomal storage diseases.

### FIELD OF THE INVENTION

The present invention relates to methods for improved treatment of lysomal storage diseases and other medical conditions. The methods include both methods of enzyme replacement therapy and gene therapy.

### BACKGROUND OF THE INVENTION

### Lysosomal Storage Diseases

Several of the over thirty known lysosomal storage diseases (LSDs) are known to involve a similar pathogenesis, namely, a compromised lysosomal hydrolase. Generally, the activity of a single lysosomal hydrolytic enzyme is reduced or lacking altogether, usually due to inheritance of an autosomal recessive mutation. As a consequence, the substrate of the compromised enzyme accumulates undigested in lysosomes, producing severe disruption of cellular architecture and various disease manifestations.

Gaucher's disease is the oldest and most common lysosomal storage disease known. Type 1 is the most common among three recognized clinical types and follows a chronic course which does not involve the nervous system. Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazim (see generally Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W. B. Saunders Co., pages 1461-1507). Also known as glucosylceramide lipidosis, Gaucher's disease is caused by inactivation of the enzyme glucocerebrosidase and accumulation of glucocerebroside. Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme sphingomyelinase in Types A and B disease, with 27-fold more residual enzyme activity in Type B (see Kolodny et al., 1998, Id.). The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosylceramide (GL-3) (see generally Desnick R J et al., 1995, α-galactosidase A Deficiency: Fabry Disease, In: The Metabolic and Molecular Bases of Inherited Disease, Scriver et al., eds., McGraw-Hill, New York, 7^{th} ed., pages 2741-2784). Symptoms may include anhidrosis (absence of sweating), painful fingers, left ventricular hypertrophy, renal manifestations, and ischemic strokes. The severity of symptoms varies dramatically (Grewal RP, 1994, Stroke in Fabry's Disease, J. Neurol. 241, 153-156). A variant with manifestations limited to the heart is recognized, and its incidence may be more prevalent than once believed (Nakao S, 1995, An Atypical Variant of Fabry's Disease in Men with Left Ventricular Hypertrophy, N. Engl. J. Med. 333, 288-293).

Recognition of unusual variants can be delayed until quite late in life, although diagnosis in childhood is possible with clinical vigilance (Ko YH et al., 1996, Atypical Fabry's Disease—An Oligosymptomatic Variant, Arch. Pathol. Lab. Med. 120, 86-89; Mendez MF et al., 1997, The Vascular Dementia of Fabry's Disease, Dement. Geriatr. Cogn. Disord. 8, 252-257; Shelley ED et al., 1995, Painful Fingers, Heat Intolerance, and Telangiectases of the Ear: Easily Ignored Childhood Signs of Fabry Disease, Pediatric Derm. 12, 215-219). The mean age of diagnosis of Fabry disease is 29 years. Replacement of the defective enzyme is considered feasible using a recombinant retrovirus carrying the cDNA encoding α-Gal A to transfect skin fibroblasts obtained from Fabry patients (Medin JA et al., 1996, Correction in Trans for Fabry Disease: Expression, Secretion, and Uptake of α-Galactosidase A in Patient-Derived Cells Driven by a High-Titer Recombinant Retroviral Vector, Proc. Natl. Acad. Sci. USA 93, 7917-7922).

### SUMMARY OF THE INVENTION

Accordingly, the present invention provides methods for the treatment of lysosomal storage diseases and other conditions. The methods may comprise therapy to deplete macrophages or Kupffer cells as a therapy or adjunct therapy for eliminating lysosomal storage products which might otherwise be stored in macrophages and/or Kupffer cells. In addition or as an alternative, macrophage depletion or apoptosis of macrophages or Kupffer cells may be performed prior to or contemporaneously with enzyme replacement therapy and/or gene therapy which utilize DNA molecules which encode a therapeutic protein of interest, such as glucocerebrosidase or sphingomyelinase, under conditions suitable for the expression of said DNA molecule.

Thus, in certain embodiments, the present invention comprises methods of treating a patient suffering from accumulation of a metabolite within macrophages, said method comprising treating the patient with a macrophage depleting amount of a bisphosphonate compound, such that apoptosis of macrophages is induced and the metabolite is released into circulation so that it may be eliminated from the patient. In preferred embodiments, the patient may be suffering from Gaucher's disease, in which case the metabolite is GL1; or the patient may be suffering from Niemann-Pick disease, in which case the metabolite is sphingomyelin. In further preferred embodiments, the methods further comprise administering to the patient a composition of purified recombinant protein. For patients with Gaucher's disease, the recombinant protein is recombinant glucocerebrosidase. For patients with Niemann-Pick disease, the recombinant protein is recombinant acid sphingomyelinase.

In other embodiments, the present invention comprises methods of treating a patient suffering from accumulation of a metabolite within macrophages, said method comprising treating the patient with a macrophage depleting amount of a bisphosphonate compound, such that apoptosis of macrophages is induced, and administering to the patient a gene therapy vector encoding a compound which is able to break down the accumulated metabolite.

In preferred embodiments, the patient may be suffering from Gaucher's disease, and the gene therapy vector encodes glucocerebrosidase. Optionally, the method may further comprise administering to the patient a composition of purified recombinant glucocerebrosidase. In embodiments wherein the patient is suffering from Niemann-Pick disease, the gene therapy vector encodes acid sphingomyelinase, and the method may optionally further comprise administering to the patient a composition of purified recombinant acid sphingomyelinase.

In still other embodiments of the present invention, the patient is suffering from Fabry's disease, and the gene therapy vector encodes alpha galactosidase A. Optionally, the method may further comprise administering to the patient a composition of purified recombinant alpha-galactosidase. In yet other embodiments, the patient is suffering from Pompe disease, and the gene therapy vector encodes alpha glucosidase. Optionally, the method may further comprise administering to the patient a composition of purified recombinant alpha glucosidase. In another embodiment, the patient is suffering from Hurler's Disease (MPS-I), and the gene therapy vector encodes alpha-L iduronidase. Optionally, the method may further comprise administering to the patient a composition of purified recombinant alpha-L iduronidase.

In the most preferred embodiments, the macrophage depleting compound is a bisphosphonate, such as clodronate, alendronate, pamidronate, zoledronate, etidronate, ibandronate, olpadronate, risedronate, medronate, neridronate, tiludronate, or incadronate. Other apoptosis inducing agents may be used, appropriately targeted to macrophages, such as through encapsulation in liposomes. In addition, the methods of the present invention may employ other compounds which will exhibit a macrophage-depleting or macrophage-inhibiting effect. By “macrophage-depleting” or “macrophage-inhibiting” effect, it is meant that the compound is present in sufficient amount to alter or block the activity of these cells and to thereby reduce or eliminate endocytosis or phagocytosis. Such compounds or substances may include, for example, doxirubicin, gamma globulin, heavy metal salts and molecules which may block the Fc receptor.

The present methods have important advantages for the treatment of lysosomal storage diseases. First, the methods of the present invention allow the persistent expression of therapeutic levels of lysosomal storage enzymes to be produced from gene therapy vectors. Second, the methods of the present invention allow for the elimination of significant amounts of lysosomal storage products which might otherwise be sequestered within macrophages and/or Kupffer cells. Third, the methods of the present invention may allow for more effective treatment of lysosomal storage diseases using enzyme replacement therapy and/or gene therapy in which lower dosage regimens may conveniently be used.

The methods of the present invention are particularly adapted towards the treatment of lysosomal storage diseases in which the lysosomal storage product is typically concentrated within macrophages. It is believed that by depleting the macrophages, the accumulated lysosomal storage product will enter the system and to a significant extent will be eliminated from the system through a combination of secretion in bile and excretion in urine, rather than stored in the macrophages. These include Gaucher's disease and Niemann-Pick Disease. Although the associated lysosomal enzymes are not as strongly localized to macrophages in other lysosomal storage diseases, such treatment of such diseases may be enhanced by the methods of the present invention. These include LAL, Pompe's (alpha-glucosidase), Hurler's (MPS I) (alpha-L iduronidase), Fabry's (alpha-galactosidase), Hunters (MPS II) (iduronate sulfatase), Morquio Syndrome (MPS IVA) (galactosamine-6-sulfatase), MPS IVB, (beta-galactosidase) and Maroteux-Lamy C (MPS VI)(arylsulfatase B). In addition, any disease in which an unwanted metabolite is accumulated within macrophages or Kupffer cells, or any therapy for which depletion of macrophages might otherwise be desired, may be a suitable candidate for therapy using the methods of the present invention.

The preferred coding DNA sequences useful for gene therapy targeting to the liver via depletion of macrophages include DNA sequences which encode a therapeutic protein for which activity and targeting to liver is desired. By depletion of macrophages, and particularly Kupffer cells, it is believed that more of the gene therapy vector can be taken up by hepatocytes, where they can efficiently produce higher levels of therapeutic protein. In particular, preferred coding DNA sequences include those sequences encoding, glucocerebrosidase and acid sphingomyelinase, for the treatment of patients with Gaucher's Disease and Niemann-Pick Disease, respectively. Other preferred coding DNA sequences include those encoding alpha-glucosidase (Pompe's Disease), alpha-L iduronidase (Hurler's Disease or MPS I), alpha-galactosidase (Fabry Disease), iduronate sulfatase (Hunters Disease (MPS II), galactosamine-6-sulfatase (MPS IVA), beta galactosidase (MPS IVB) and arylsulfatase B (MPS VI).

As described further herein, the methods of the present invention, which utilize macrophage-depleting agents such as clodronate, the macrophage-depleting agent may be utilized in combination with other agents which have a macrophage-depleting or macrophage-inhibiting effect. By macrophage-inhibiting effect, it is intended to mean that the substance is able to interact with macrophages, either via the Fc receptor or other means, and is thereby able to reduce or eliminate the activity of such macrophages in endocytosis or phagocytosis.

In either aspect of the present invention, treatment with clodronate or other macrophage-depleting substance, may be useful in conjunction with more traditional therapies, such as enzyme-replacement therapy. Thus, for the treatment of Gaucher disease, the methods of the present invention may be used in addition to treatment with recombinantly produced glucocerobrosidase, commercially available as Cerezyme® [Genzyme Corporation, Cambridge, Mass.; also see U.S. Pat. No. 5,236,838]. For treatment of Fabry disease, the methods of the present invention may be used in addition to treatment with recombinantly produced alpha-galactosidase [see U.S. Pat. No. 5,580,757]. Use of the methods of the present invention may allow for the use of lower doses, or less frequent dosing, with enzyme replacement therapy.

### BRIEF DESCRIPTION OF THE FIGURES

FIG. 1: Enhanced expression is generated from a low dose of virus by pre-treating mice with clodronate liposomes to deplete Kupffer cells in the liver.

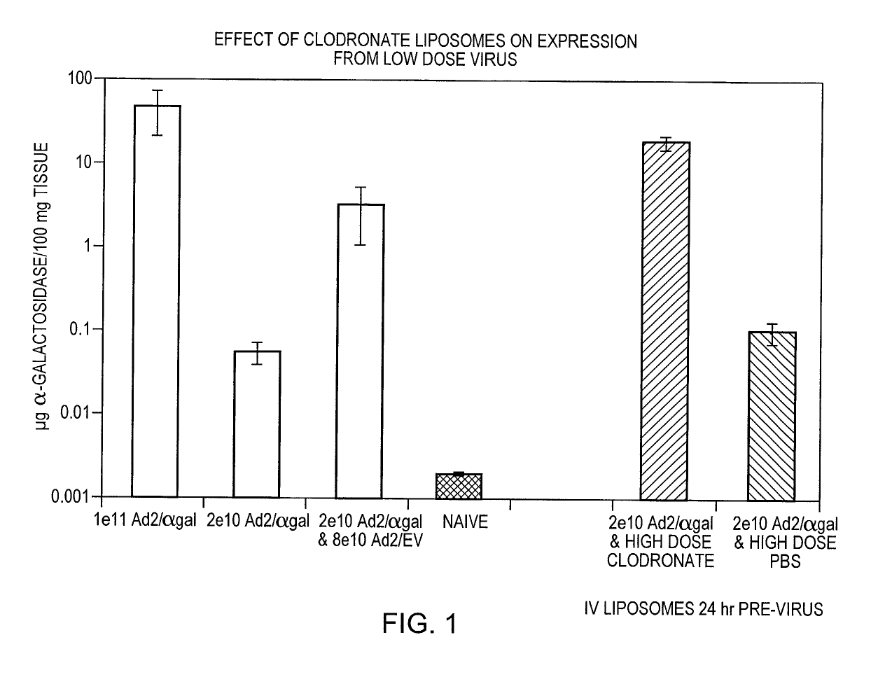

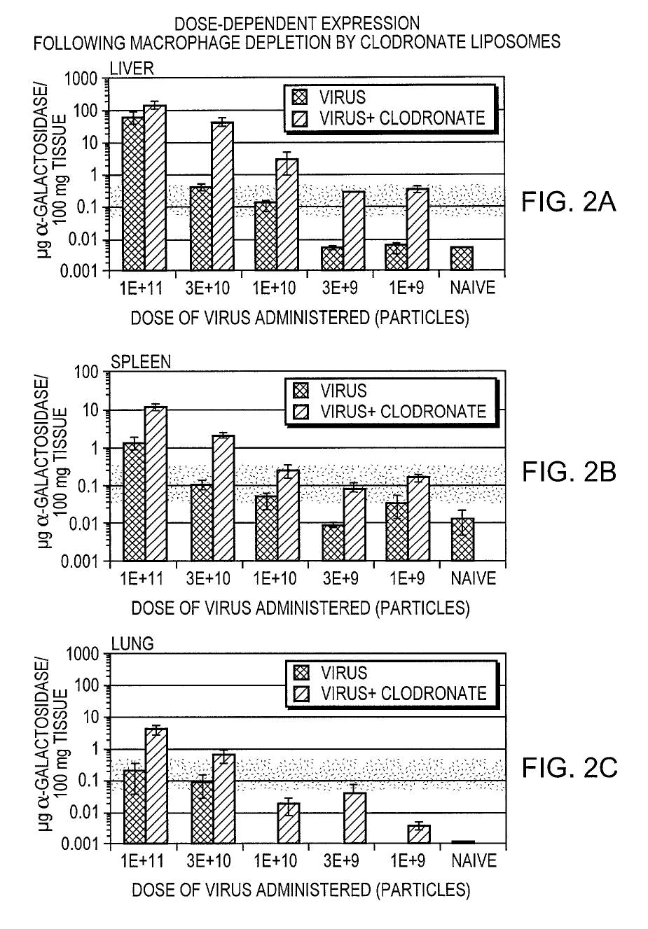

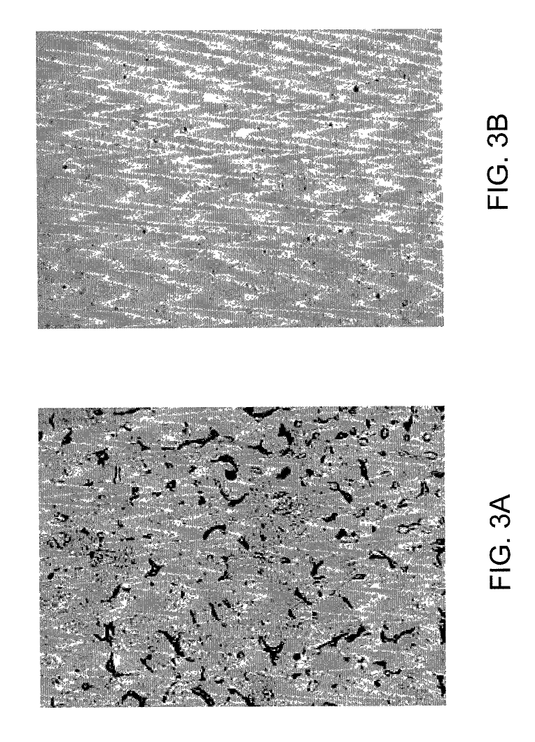

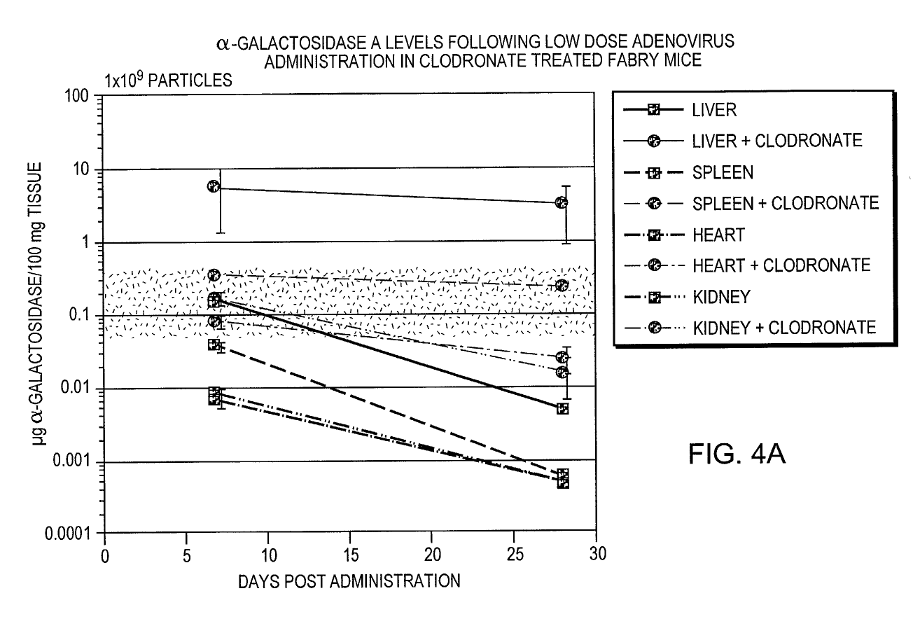

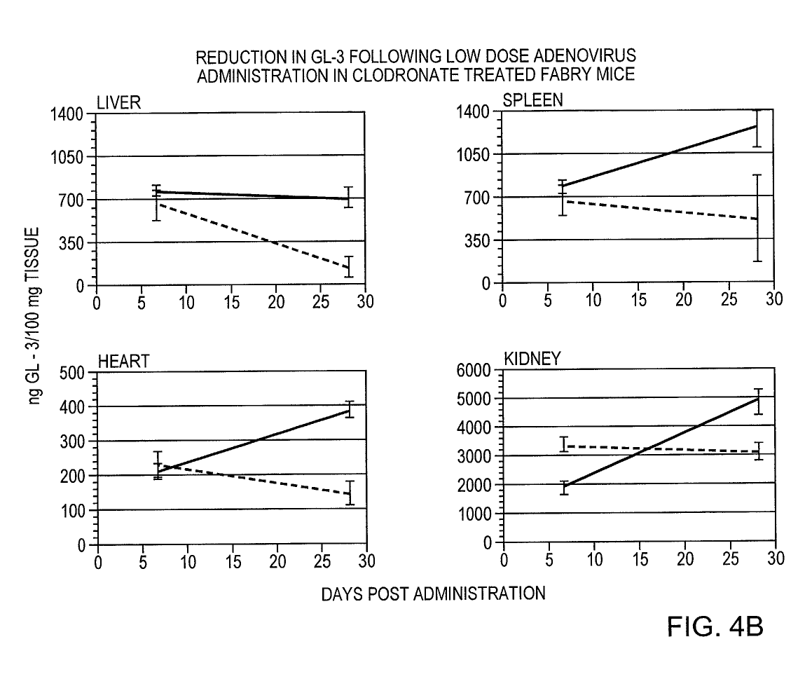

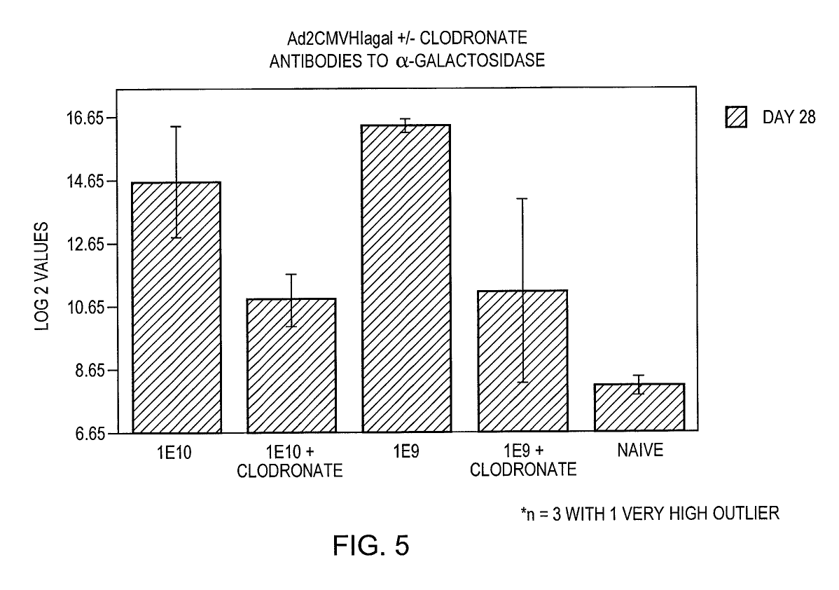

Groups of four mice were given the following three treatment regimes: A) 1×10¹¹ particles of Ad2/CMVHIαgal, B) 2×10¹⁰ particles of Ad2/CMVHIαgal, C) 2×10¹⁰ particles of Ad2/CMVHIαgal mixed with 8×10¹⁰ particles Ad2/EV. Two additional groups were pre-treated 24 hours prior to administration of a low dose of virus, one with 50 μl clodronate liposomes to deplete macrophages in the liver (Kupffer cells), the other with 50 μl PBS liposomes as a control. Mice were sacrificed three days following virus administration. Liver homogenates were analyzed for α-galactosidase A expression using an ELISA specific for human α-galactosidase A. Expression from a low dose of Ad2/CMVHIαgal was enhanced in mice pre-treated with clodronate liposomes. The effect was similar to that seen in the group supplemented with empty vector.

FIG. 2: Dose-dependant expression is achieved following macrophage depletion by clodronate liposomes.

Groups of BALB/c mice were injected with various doses of Ad2/CMVHIαgal as described in the graph, 24 hours prior to virus administration one half of the mice at each dose of the vector had been treated with 50 μl of clodronate liposomes to deplete macrophages in the liver (Kupffer cells). Tissue homogenates were analyzed for α-galactosidase A expression using the ELISA (the shaded area with the graph represents the range of α-galactosidase A in normal mouse tissues). Three tissues were evaluated for expression as shown. Mice treated with clodronate liposomes showed higher levels of expression at all doses compared to mice receiving virus alone. The expression levels were closely correlated with the dose of virus administered. Therapeutic levels of α-galactosidase A were attained with a 50-fold lower dose of virus after clodronate liposome treatment.

FIG. 3: Immunohistochemical staining of liver sections from mice treated with clodronate liposomes.

Mice from the experiment described in FIG. 2 were perfused with 10% neutral-buffered formalin upon sacrifice, the livers removed and fixed for 24 hours. The samples were embedded in paraffin and 0.5 μm sections cut. Sections were deparaffinized, rehydrated and immunohistochemistry performed. Sections were stained with a rat monoclonal antibody to the mouse macrophage surface marker F4/80 (Serotec) using the VectaStain ABC kit from Vector Laboratories. Slides were counterstained from hematoxylin and visualized at 200×magnification. Macrophages can be identified by the dark brown stain. Kupffer cells were almost completely depleted in the livers of mice treated with clodronate liposomes.

FIG. 4: Improved persistence and efficacy from a reduced dose of adenovirus following macrophage depletion

1×10⁹ particles of virus were injected into the tail vein of two groups of Fabry mice. One group had been pre-treated with clodronate liposomes. Organs were divided to be assayed for both α-galactosidase A expression and GL-3 levels. Tissues were assayed by ELISA. FIG. 4A shows α-galactosidase A levels. The shaded area within the graph represents the range of α-galactosidase A in normal mouse tissues. FIG. 4B shows GL-3 levels. The dashed lines represent mice pre-treated with clodronate liposomes, and the solid lines represent mice treated with virus alone. Values represent an average of four treated mice per group. Clodronate liposome pre-treatment enhanced levels and persistence of expression from 1×10⁹ particles of Ad2/CMVHIαgal with resulting GL-3 clearance in all tissues except kidney. This dose of vector was not sufficient to clear GL-3 in Fabry mice treated with virus alone.

FIG. 5: Antibody levels in plasma from Fabry mice treated with Ad2/CMVHIαgal +/−clodronate liposomes

Antibody titers were measured in the plasma samples collected from the animals described in FIG. 4. Antibodies made to human α-galactosidase were assayed using an ELISA format. Symbols represent titers from individual animals. Fabry mice pre-treated with clodronate liposomes made significantly lower levels of antibodies to α-galactosidase A than mice treated with adenovirus alone.

### DETAILED DESCRIPTION OF THE INVENTION

It is known that many of the lysosomal enzymes, and in particular, glucocerebrosidase (Gaucher's Disease) and acid sphingomyelinase (Niemann-Pick Disease A & B), are particularly good candidates for methods of treatment involving depletion of macrophages, since the lysosomal storage products involved in these diseases in large part accumulate in the macrophages and Kupffer cells.

Other lysosomal storage disorders which may be suitable for gene therapy avoidance of macrophages and Kupffer cells include LAL, Pompe's (alpha-glucosidase), Hurler's (alpha-L iduronidase), Fabry's (alpha-galactosidase), Hunters (MPS II) (iduronate sulfatase), Morquio Syndrome (MPS IVA)(galactosamine-6-sulfatase), MPS IVB (beta-D-galactosidase), and Maroteux-Lamy C (MPS VI)(arylsulfatase B).

There is evidence of other independent pathways, in addition to the mannose-6-phosphate pathway, that may function in the transport of lysomal enzymes inside cells and of alternate mechanisms for the internalization of lysosomal enzymes by cell-surface receptors in addition to mannose-6-phosphate receptors (Scriver et al. 1995). In addition, any protein for which avoidance of the macrophages is desired may be a suitable candidate for the method of the present invention.

In certain embodiments of the present invention, purified recombinant proteins are produced in cell culture, using coding DNA sequences to transduce cells, such as CHO cells, yeast cells, or other cells suitable for recombinant protein production. In other embodiments of the present invention, nucleotide sequences encoding a lysosomal enzyme or other therapeutic protein are provided via administration of gene therapy vectors. Such gene therapy vectors include recombinant viral vectors containing coding DNA sequences, in which aspects of viruses are used to transfect cells, which then will express the coding DNA sequences to produce the encoded lysosomal enzyme or therapeutic protein. Suitable virus systems are known for example, for adenovirus, adeno-associated virus and other parvoviruses, alphaviruses, and retroviruses, including lentiviruses. Other gene therapy vectors include non-viral systems for transducing cells, such as the use of cationic lipids or other cationic molecules as the cell-transduction mediator. Also known in the art are methods for delivery of naked DNA into cells.

The preferred coding DNA sequences include sequences encoding any therapeutic protein. In preferred embodiments, the coding DNA sequence is one which is desired to be targeted to hepatocytes, without accumulation in macrophages. In particular, preferred coding DNA sequences include those sequences encoding, glucocerebrosidase for the treatment of patients with Gaucher's Disease and acid sphingomyelinase for the treatment of patients with Niemann-Pick Disease, respectively. Other preferred coding DNA sequences include those encoding alpha-glucosidase (Pompe's Disease), alpha-L iduronidase (Hurler's Disease), alpha-galactosidase (Fabry's Disease), and iduronate sulfatase (Hunters Disease (MPS II), galactosamine-6-sulfatase (MPS IVA); beta-D-galactosidase (MPS IVB); and arylsulfatase B (MPS VI). In addition, a cDNA for any protein to be expressed from liver, for which avoidance of the macrophages is desired may be a suitable candidate for the methods of the present invention.

Methods for the purification of recombinant human proteins are well-known, including methods for the production of recombinant human glucocerebrosidase [for Gaucher's Disease]; sphingomyelinase [for Niemann-Pick Disease], alpha-galactosidase [for Fabry Disease]; alpha-glucosidase [for Pompe's Disease]; alpha-L iduronidase [for Hurler's Syndrome]; iduronate sulfatase [for Hunter's Syndrome]; galactosamine-6-sulfatase [for MPS IVA]; beta-D-galactosidase [for MPS IVB]; and arylsulfatase B [for MPS VI]. See, for example, Scriver et al., eds., The Metabolic and Molecular Bases of Inherited Diseases, Vol. II., 7^{th} ed. (McGraw-Hill, N.Y.; 1995), the disclosure of which is hereby incorporated herein by reference.

Compounds which may be useful in the methods of the present invention include compounds which are able to induce apoptosis of macrophages and Kupffer Cells. Such compounds include members of the bisphosphonate family, such as clodronate, alendronate, pamidronate, zoledronate, etidronate, ibandronate, olpadronate, risedronate, medronate, neridronate, tiludronate, and incadronate. [See Rogers et al., Cancer, 88/12 Suppl. 2961-2978 (2000)]. Other apoptosis-inducing agents which may be useful in the methods of the present invention include Fas ligand [see Watanabe-Fukunaga et al., Nature 314-317, 1992; Zhou et al., J. Exp. Med. 176:1063-1072, 1992; Nagata, Adv. Immunol. 57:129-144, 1994, and Dhein et al., Nature 373:438-441, 1995]. In general, a wide range of apoptosis inducing agent concentrations can be used. Van Rooijen and Sanders, (1994) J. Immunological Methods 174:83-93, identifies preferred concentrations for clodronate encapsulated in liposomes. See also, Van Rooijen and Sanders (1996) Hepatology 23:1239-1243; Wang et al (1999) Oral Pathol. Med. 28:145-151; Schugart et al. (1999) Gene Therapy, 6:448-453; Lieber et al. (1997), J. Virology, 71:8798-8807; Kuzmin et al. (1997) Gene Therapy, 4:309-316; Worgall et al. (1997), Human Gene Therapy, 8:1675-1684; Stein et al. (1998), Gene Therapy, 5:431-439; Wolff et al. (1997) J. Virology, 71:624-629; Van Rooijen et al. (1996) J. Immunological Methods, 193:93-99; and Van Rooijen and Sanders (1997) Trends in Biotechnology, 15:178-185.

### Treatment of Gaucher's Disease and Niemann-Pick Disease—Apoptosis of Kupffer Cells

Therapy for metabolic storage diseases of macrophages [e.g., Gaucher disease] may be accomplished by the use of agents that disrupt macrophages, such as bisphosphonates. In Gaucher disease, the primary clinical manifestation is massive accumulation of glucosyl ceramide, or GL1, the substrate for the enzyme glucocerebrosidase [GCR], in the macrophages, particularly of the liver or the spleen. Induction of apoptosis in these macrophages, by treatment with bisphosphonates such as clodronate or compounds with similar effects would cause release of the accumulated glycolipid. This could allow for removal of GL1 from the body through bile salt secretion and/or urine excretion. Circulating GL1 may also re-distribute into cell types which are more readily accessible to treatment by enzyme replacement therapy and/or gene therapy either by direct administration into and/or transduction of the affected cells, or by the affected cells' ability to take up circulating mature GCR secreted by a distantly transduced site [e.g., a targeted cell or depot organ].

Circulating GL1 must be primarily reabsorbed by hepatocytes in order for it to be targeted for biliary secretion. Indications are that the majority of a bolus injection of liposome-encapsulated GL1 ends up in the liver parenchyma. Pentchev et al., BBA, (1981); Tokoro et al., J. Lip. Res., (1987); Hers, Gastroenterology 48:625-632 (1965). An additional amount is found in Kupffer Cells. It appears that rapid influx of circulating GL1 storage product, and the loss of liver Kupffer Cells are not harmful to patients. As a result, it is predicted by the inventors that GL1can be eliminated from the body of Gaucher's Disease patients by apoptosis of the Kupffer Cells, potentially in combination with enzyme replacement treatment and/or gene therapy treatment with GCR.

Similarly, Niemann-Pick disease mice exhibit elevated levels of stored lipidic storage products in Kupffer Cells. Accordingly, apoptosis of Kupffer Cells may similarly be useful for treatment of Niemann-Pick disease in human patients. Such treatment may be accomplished using clodronate or other bisphosphonates, as well as Fas ligand targeted to Kupffer Cells. For treatment of Niemann-Pick, such treatment can be accomplished in combination with enzyme replacement treatment and/or gene therapy treatment with sphingomyelinase [see U.S. Pat. Nos. 5,686,240; 5,773,278].

### Improving Efficacy of Viral-mediated Gene Therapy of Lysosomal Storage Disorders: Increased Expression from a Reduced Dose of Vector by Avoidance of Macrophages in the Transduced Depot Organ

Treatment of other lysosomal storage disorders, such as Fabry Disease, may require significant transduction of a target depot organ, such as liver or lung. Secretion of the therapeutic transgene is driven by overexpression. Secretion into circulation and subsequent uptake by distal diseased tissues is required for efficacy of this treatment. We have shown in Fabry mice that a high dose of adenoviral vector (10¹¹ particles) was required for efficient clearance of the lipidic substrate deposited in diseased tissues, globotriaosylceramide, or GL-3. This dose of adenovirus can be associated with significant liver toxicity. Reducing the dose of adenovirus particles resulted in a non-linear dose response in expression; a ten-fold reduction in dose yielded a 150 fold drop in expression. Treating mice systemically with clodronate liposomes depleted macrophages from the liver and boosted the level of expression, thus therapeutic levels of expression were attained from a 50-fold lower dose of virus. We propose that this effect can extend to other therapeutic proteins for lysosomal storage diseases and other diseases; to other depot organs, such as lung; as well as to other gene therapy vectors, such as adeno-associated virus, other viral-based gene therapy vectors, and non-viral gene therapy vectors. Clodronate liposome treatment, or any other method for circumventing macrophage uptake of vectors, should allow us to treat effectively with a lower, less toxic dose of gene therapy vector.

Fabry mice are pre-treated with liposomes 24 hours prior to gene therapy vector administration to deplete macrophages. ‘Liposomes’ may comprise:

a. Clodronate liposomes prepared as described. Van Rooijen and Sanders, (1994) J. Immunological Methods 174:83-93.

b. Liposomes encapsulating other drugs effective at depleting macrophages without activation of said macrophages.

In addition, gene therapy vectors encoding a lysosomal enzyme, such as alpha-galactosidase in the case of Fabry Disease, may be complexed with molecules (such as PEG) which further shield them from recognition and uptake by macrophages.

### Alternative or Additional Macrophage-Depletion Strategies

In addition to, or instead of, treatment with clodronate or other bisphosphonates, other methods are available which may prove useful for the depletion of macrophages and/or Kupffer cells, and thus for reducing or avoiding the immune response to gene therapy, in accordance with the methods of the present invention. These include use of other macrophage-depleting substances, such as doxorubicin encapsulated in liposomes, which has been demonstrated to have a macrophage depleting effect. Daemen et al, Int. J. Cancer. 61:716-721 (1995). Doxorubicin, which is approved for cancer therapy, has some associated toxicities. However, by using lower and less frequent doses, it may be possible to achieve a macrophage depleting effect which aids in prolonged expression from gene therapy vectors, without severe adverse effects. Other molecules which might be useful for methods of the present invention include molecules which may specifically induce apoptosis or ablation of macrophages or Kupffer cells. This may include Fas ligand or other apoptotic or ablative agents targeted to macrophages. One preferred method for targeting macrophages is encapsulation in liposomes.

As described further herein, the methods of the present invention, which utilize macrophage-depleting agents such as clodronate, the macrophage-depleting agent may be utilized in combination with other agents which have a macrophage-depleting or macrophage-inhibiting effect. By macrophage-inhibiting effect, it is intended to mean that the substance is able to interact with macrophages, either via the Fc receptor or other means, and is thereby able to reduce or eliminate the activity of such macrophages in endocytosis or phagocytosis.

In either aspect of the present invention, treatment with clodronate or other macrophage-depleting substance, may be useful in conjunction with more traditional therapies, such as enzyme-replacement therapy. Thus, for the treatment of Gaucher disease, the methods of the present invention may be used in addition to treatment with recombinantly produced glucocerobrosidase, commercially available as Cerezyme® [Genzyme Corporation, Cambridge, Mass.; also see U.S. Pat. No. 5,236,838]. For treatment of Fabry disease, the methods of the present invention may be used in addition to treatment with recombinantly produced alpha-galactosidase [see U.S. Pat. No. 5,580,757]. Use of the methods of the present invention may allow for the use of lower doses, or less frequent dosing, with enzyme replacement therapy.

As an alternative, or additional, strategy to effectively deplete macrophages or Kupffer cells, approaches can be used which will alter or block the activity of these cells to thereby reduce or eliminate endocytosis or phagocytosis. A number of drugs are available in this regard. Most notably, human gamma globulins (IG) may be used. This class of drugs is presently approved product for supplementing immune system. While the exact mechanism of action is not determined, it is believed that these drugs can essentially swamp the immune system by forming a non-specific reticuloendothelial system blockade. See Samuelsson et al., Science 291:484-486 (2001).

Methods of the invention may further comprise the use of other methods include the use of molecules which may be able to block or mask the Fc receptors on macrophages. Such molecules may include heavy metal salts, such as gadolinium chloride [see Husztik et al., Brit. J. Exper. Path., 61:624-30 (1980); and Hardonk et al., J. Leukocyte Biol., 52:296-302 (1992)], derivatives of deoxynojirimycin [DNJ] [see Overkleef et al., J. Biol. Chem., 273:26522-26527 (1998); WO98/02161] a sugar analog inhibitor of glucosylceramide synthase; and mannin, which is essentially a polymer of numerous mannose entities. Other such substances may include large neutral polymers such as polyethylene glycol (PEG) or hyaluronic acid (HA) polymers.

While the invention is exemplified with respect to the production of specific proteins, these examples are not to be interpreted as limiting the invention in any manner. The invention is further not limited to any stated belief as to the mechanism for action. As described above, and as will be clear to those skilled in the art from reading the specification, the methods of the present invention are useful for the treatment of lysosomal storage diseases, and for a number of other conditions which may involve accumulation of unwanted substances within macrophages, including the lysosomal storage products described above. Many modifications and variations of the methods and materials used in the present description will also be apparent to those skilled in the art. Such modifications and variations fall within the scope of the invention.

The entire disclosures of all of the publications and references cited in this specification are hereby incorporated herein by reference.

### EXAMPLES

### Viral-mediated Gene Therapy of Fabry Disease.

Fabry disease is caused by a deficiency of the lysosomal hydrolase α-galactosidase A.

This results in progressive accumulation of globotriaosylceramide (GL-3) in lysosomes. The feasibility of using gene therapy for treating Fabry disease has been demonstrated. Intravenous administration of Ad2/CMVHIαgal into Fabry mice resulted in correction of both the enzymatic and lysosomal storage defects in all affected organs. However there was associated liver toxicity and expression subsided over several months. The methods of the present invention will be useful in combination with more advanced versions of adenoviral vectors with improved toxicity profiles, as well as strategies to enhance delivery of the virus to hepatocytes. In order to moderate toxicity, we sought to lower the dose of vector administered. However, reducing the dose of Ad2/CMVHIαgal by ten fold resulted in a greater than 200 fold drop in expression of α-galactosidase A, a level that was insufficient to completely clear accumulated GL-3. In order to address this problem, mice were pre-treated with clodronate liposomes to deplete Kupffer Cells. Administration of 2×10¹⁰ particles of Ad2/CMVHIαgal in the presence of clodronate liposomes generated higher expression levels of α-galactosidase A than in control animals that were treated with PBS liposomes. The level of α-galactosidase A attained in the presence of clodronate was consistent with the dose used. This suggests that sequestration of viral particles by the Kupffer cells may play a role in the non-linear dose response. The use of clodronate liposomes resulted in lower levels of antibodies forming to human α-galactosidase A. Using the lower doses of adenoviral particles, which are effective persistent expression with administration of clodronate-containing liposome, results in reduced levels of cytokines, such as IL-6 and IL-12, which are associated with toxicity of high doses of adenoviral particles. Hence, strategies such as the use of clodronate, for the depletion of Kupffer cells to prevent this process, may allow the use of lower therapeutic doses of gene therapy vectors for the treatment of lysosomal storage diseases, such as Fabry, and ultimately safer gene therapy treatment.

An alternative viral vector reported to show lower toxicity and more persistent expression is adeno-associated virus (AAV). Systemic administration of AAV/CMVHIαgal into immune-suppressed Fabry mice generated a-galactosidase A levels in the liver that were approximately 10% of normal. These levels were undiminished over three months. Although expression was significantly lower than the levels obtained from adenovirus vectors, GL-3 was reduced in all tissues assayed except kidney. Thus, depletion of macrophages using the methods of the present invention may also be applicable to AAV vectors. In addition, AAV vectors containing optimized transcription cassettes may be used in order to enhance expression to levels that are therapeutic for use in Fabry disease.

### Viral-mediated Gene Therapy of Gaucher Disease

We have used clodronate in two studies using adenoviral vectors encoding the human glucocerebrosidase (GC) gene. The experimental details are essentially the same as for αgal except we used wild-type BALB/c mice as there presently is no viable in vivo Gaucher disease model.

In the first study, we analyzed the levels of GC enzyme activity obtained from a tail vein intravenous bolus delivery of 10¹¹ virus particles in 100 μl. There were 3 groups of 4 mice (BALB/c) each. Group A: naive mice. Group B: received virus, without clodronate. Group C received a dose of clodronate the day before virus administration. Resulting expression compared to naïve was:

Group A. serum 1x; liver 1x

Group B. serum 12x (33 μg/ml); liver 3x (96 μg/gm)

Group C. serum 500x (1400 μg/ml); liver 230x (750 μg/gm)

The second experiment was a repeat of the first but included two additional groups, each group receiving a viral dose of 2×10¹⁰, Group D without clodronate; Group E with clodronate. Resulting expression compared to naïve was:

Group A. serum 1x; liver 1x

Group B. serum 1x; liver 9x (131 μg/gm)

Group C. serum 1600x (7520 μg/ml); liver 117x (1209 μg/gm)

Group D. serum 1x; liver 4x (58 μg/gm)

Group E. serum 117x (553 μg/ml); liver 72x (1053 μg/gm)

In summary, mice that received 10¹¹ Ad2/hGC particles exhibited liver expression of GC protein at ˜5x endogenous levels, and serum GC levels at ˜6x endogenous levels. Pre-treatment with clodronate liposomes yielded liver expression on the order of 100-fold that of endogenous GC levels and serum GC levels on the order of 1000-fold that of endogenous GC levels following the same dose of virus.

We claim:

1. A method of treating a patient suffering from accumulation of a metabolite within macrophages, said method comprising treating the patient with a macrophage depleting amount of a bisphosphonate compound, such that apoptosis of macrophages is induced and the metabolite is released into circulation so that it may be eliminated from the patient.

2. The method of claim 1, wherein the bisphosphonate compound is clodronate.

3. The method of claim 1, wherein the patient is suffering from Gaucher's disease, and the metabolite is GL1.

4. The method of claim 3, further comprising administering to the patient a composition of purified recombinant glucocerebrosidase.

5. The method of claim 1, wherein the patient is suffering from Niemann-Pick disease, and the metabolite is sphingomyelin.

6. The method of claim 5, further comprising administering to the patient a composition of purified recombinant acid sphingomyelinase.

7. A method of treating a patient suffering from accumulation of a metabolite within macrophages, said method comprising treating the patient with a macrophage depleting amount of a bisphosphonate compound, such that apoptosis of macrophages is induced, and administering to the patient a gene therapy vector encoding a compound which is able to break down the accumulated metabolite.

8. The method of claim 7, wherein the patient is suffering from Gaucher's disease, and the gene therapy vector encodes glucocerebrosidase.

9. The method of claim 8, further comprising administering to the patient a composition of purified recombinant glucocerebrosidase.

10. The method of claim 7, wherein the patient is suffering from Niemann-Pick disease, and the the gene therapy vector encodes acid sphingomyelinase

11. The method of claim 10, further comprising administering to the patient a composition of purified recombinant acid sphingomyelinase.

12. The method of claim 7, wherein the patient is suffering from Fabry's disease, and the gene therapy vector encodes alpha galactosidase A.

13. The method of claim 12, further comprising administering to the patient a composition of purified recombinant alpha-galactosidase.

14. The method of claim 7, wherein the patient is suffering from Pompe disease, and the gene therapy vector encodes alpha glucosidase.

15. The method of claim 14, further comprising administering to the patient a composition of purified recombinant alpha glucosidase.

16. The method of claim 7, wherein the patient is suffering from Hurler's Disease (MPS-I), and the gene therapy vector encodes alpha-L iduronidase.

17. The method of claim 16, further comprising administering to the patient a composition of purified recombinant alpha-L iduronidase.

18. The method of claim 7, further comprising administration of a macrophage depleting or macrophage inhibiting compound selected from the group consisting of doxicirubin, gamma globulin, and neutral polymers.
